SEQUENCE LISTING (12 sequences)

SEQ ID NO: 1 - DNA, 24 bp, Artificial Sequence
  Description of Artificial Sequence PCR-Primer
ggagtattttaatgcaagtagccc

SEQ ID NO: 2 - DNA, 20 bp, Artificial Sequence
  Description of Artificial Sequence PCR-Primer
gtagctaagggtgggcctct

SEQ ID NO: 3 - DNA, 24 bp, Artificial Sequence
  Description of Artificial Sequence PCR-Primer
gctctccgggcctcgaaagagatt

SEQ ID NO: 4 - DNA, 20 bp, Artificial Sequence
  Description of Artificial Sequence PCR-Primer
aagaggacctgggagaagct

SEQ ID NO: 5 - DNA, 21 bp, Artificial Sequence
  Description of Artificial Sequence PCR-Primer
ccttctcagcagcagccctgc

SEQ ID NO: 6 - DNA, 31 bp, Artificial Sequence
  Description of Artificial Sequence PCR-Primer
atttctcgagtgtccggagcccttgatgtcc

SEQ ID NO: 7 - DNA, 31 bp, Artificial Sequence
  Description of Artificial Sequence PCR-Primer
attgaattccttacgaccactcggtggtggt

SEQ ID NO: 8 - DNA, 30 bp, Artificial Sequence
  Description of Artificial Sequence PCR-Primer
attttctagaaacgaccactcggtggttcc

SEQ ID NO: 9 - protein, 22 aa, Artificial Sequence
  Description of Artificial Sequence Laminin Receptor Peptide
MSGALDVLQMKEEDVLKFLAGC

SEQ ID NO: 10 - protein, 22 aa, Artificial Sequence
  Description of Artificial Sequence Laminin Receptor Peptide
RLLVVTDPRADHQPLTEASYGC

SEQ ID NO: 11 - protein, 21 aa, Artificial Sequence
  Description of Artificial Sequence Laminin Receptor Peptide
KEEQAAEKAVTKEEFQGEWGC

SEQ ID NO: 12 - protein, 22 aa, Artificial Sequence
  Description of Artificial Sequence Laminin Receptor Peptide
FTAAQPEVADWSEGVQVPSVGC

(19) United States
(12) Patent Application Publication
(10) Pub. No.: US 2003/0129667 A1
(43) Pub. Date: Jul. 10, 2003

(54) Method of diagnosing transmissible spongiform encephalopathies

(75) Inventors: Matthias Giese, Heidelberg (DE); Mark Stephen Rogers, Gleyncree Wicklow (IE)

Correspondence Address: BOEHRINGER INGELHEIM CORPORATION, 900 RIDGEBURY ROAD, P. O. BOX 368, RIDGEFIELD, CT, 06877, US

(73) Assignee: Boehringer ingelheim Vetmedica GmbH, Ingelheim (DE)

(21) Appl. No.: 10/278,314

(22) Filed: Oct. 23, 2002

Related U.S. Application Data
(63) Continuation of application No. 09/547,580, filed on Apr. 12, 2000.
(60) Provisional application No. 60/131,420, filed on Apr. 28, 1999.

(30) Foreign Application Priority Data
Apr. 21, 1999 (DE) 199 18 141.1

(51) Int. Cl. G01N 33/53; G01N 33/567

(52) U.S. Cl. 435/7.2

(57) ABSTRACT

The invention relates to a method of pre-clinical and clinical diagnosis of transmissible spongiform encephalopathies, characterised in that the altered expression of a marker protein is measured. In particular embodiments, in the method according to the invention, the marker protein measured is the prion protein PrP-sen or interferon gamma (IFNγ) or the laminin receptor (LR) or the laminin receptor precursor (LRP). The invention also relates to a test kit using antibodies specific to the marker protein according to the invention. The invention further relates to a test kit using oligonucleotides which are capable of hybridising under stringent conditions with the nucleic acid coding for the marker protein according to the invention. The invention further relates to the use of antibodies or oligonucleotides which are specific for the above-mentioned marker proteins in a method according to the invention. The invention further relates to the use of the test kit for diagnosing transmissible spongiform encephalopathies.

### RELATED APPLICATIONS

This application is a continuation of U.S. application Ser. No. 09/547,580, filed Apr. 12, 2000, which claims the benefit of U.S. Provisional Application No. 60/131,420, filed Apr. 28, 1999, both of which are herein incorporated by reference.

### FIELD OF THE INVENTION

The invention relates to a method of diagnosing transmissible spongiform encephalopathies and a diagnostic test kit using prion-protein- and laminin-receptor-specific antibodies. The invention also relates to the use of the method or test kit for diagnosing transmissible spongiform encephalopathies.

### BACKGROUND OF THE INVENTION

Over 250 years ago a disease of sheep was discovered which was accompanied by nervousness, itching and ataxia and finally ended in paralysis and death. This disease is now known as “Scrapie” in English speaking countries (as the animals rub against posts and trees in order to control the itching), “la tremblante” in French and “Traberkrankheit” in German. These names reflect the variety of the symptoms. “Scrapie” was investigated as the prototype of a group of diseases which affect not only animals but also human beings: the transmissible spongiform encephalopathies (=TSE). TSEs are fatal neurogenerative diseases which can attack a number of mammals.

Bovine spongiform encephalopathy (BSE) is a neurodegenerative disease in cattle and is related to Scrapie in sheep and goats and Creutzfeldt-Jakob disease in humans. A host-coded, membrane-associated glycoprotein of unknown function, the so-called prion protein PrP, plays a central part in the pathogenesis of these diseases. This cellular isoform is expressed particularly strongly on neuronal cells but can also be detected with variable frequency on non-neuronal cells. This membrane protein is sensitive (PrP-sen) to digestion with specific enzymes (proteases). However, the soluble malignant form of PrP (PrP-res) is protease-resistant and accumulates in the brains of BSE-infected animals to form amyloid plaques. This PrP-res form is associated exclusively with all TSE diseases including BSE and can be extracted from TSE/BSE-infected brain tissue.

The unusual characteristics of the Scrapie/BSE pathogen gave rise to speculation at an early stage that the pathogen consists solely of nucleic acid or proteins or contains neither nucleic acid nor proteins and is a polysaccharide or a membrane fragment. The scenarios which are most discussed at present are the “protein only ” hypothesis and the virus/virino hypothesis:

The “protein only ” hypothesis is based on the infectious prion PrP-res containing no nucleic acid and being self-replicating. It is speculated that PrP-res binds to PrP-sen and thereby converts it into the malignant isoform. The conformation of this malignant isoform is marked by beta-pleated sheet structures, whereas in the cellular isoform the alpha helices predominate. The virus/virino hypothesis is based on the infectious agent consisting of viral nucleic acid (possible RNA) and the prion protein being a shell for the virus genome. The host origin of the prion shell would explain the absence of immunological and inflammatory reactions. The existence of a nucleic acid would additionally explain the 20-odd different Scrapie-mouse strains which have been described hitherto.

The cellular isoform PrP-sen is glycosylated at two asparagine positions, has a molecular weight of 33-35000 Da and is anchored to the outer surface of the plasma membrane by a phosphatidyl-inositol glycolipid which is fixed at its carboxy-terminal amino acid. The highest expression rate of PrP-sen is measured in the brain, but the gene is also expressed in non-neuronal embryonic and adult tissue. The biological function of the protein is still unclear today. It is thought, inter alia, that PrP might be a receptor for neurotrophic differentiation factors. This protein has also been linked to the sleeping/waking rhythm. However, PrP could also be a receptor for neurotrophic viruses.

The normal cellular isoform of the protein is totally degraded by proteases (PrP-sen). The malignant isoform (PrP-res), on the other hand, is degraded by proteases into a 27-30000 Da fragment which is still completely infectious (PrP-res). The PrP-res lacks the first 67 amino acids of the mature PrP-sen protein. A post-transcription process is connected with the conversion of PrP-sen to PrP-res, and it is suspected that the only difference between PrP-sen and PrP-res is a difference in the three-dimensional structure. There does not appear to be any biochemical difference between the normal and abnormal form of the protein. This also explains why the two isoforms do not display any antigenic difference. Moreover, PrP-sen and PrP-res have a common amino acid sequence. PrP is coded by a single copy of a chromosomal gene and is highly conserved in mammals. The entire PrP-coding sequence is contained in a single exon.

In contrast to PrP-sen, which is expressed on the surface, PrP-res accumulates in cytoplasmic vesicles, many of which are secondary lysosomes.

TSE diseases are characterised by a long incubation period during which no clinical symptoms are observed. This is followed by a short clinical phase which invariably leads to death.

Scrapie and BSE have hitherto been diagnosed using histopathological, clinical and epidemiological methods, since naturally infected animals probably do not react serologically to PrP-res and diagnosis by inoculation into laboratory animals can take up to 18 months. Clinical diagnosis is made post-mortem by histopathological examination of the brain. The BSE status is subdivided into: BSE-positive, BSE-negative and BSE-suspected. Animals regarded as BSE-suspected display the same clinical signs as BSE-positive animals. However, at the time of examination, the histopathological evidence of BSE is (still) negative. However, this “BSE-suspected” state may be a “pre-BSE-positive” state, though in some cases an alternative diagnosis is made or the animal may not have BSE.

The complex diagnostic methods mentioned above contain the microscopic investigation of, for example, BSE-specific vacuoles in the neurons and neuropil, astrocytosis, neuronal loss and the depositing of abnormal accumulations of PrP-res, also known as Scrapie-associated fibrils (SAF). SAF can be detected in situ by immunohistochemistry of histoblots and in treated extracts of the affected brain by Western blotting, dot blots or as typical fibril accumulations by negative staining in transmission electron microscopy.

Another approach for diagnosing BSE indirectly is by analysing cerebrospinal fluid. Neurological diseases are associated with qualitative and quantitative changes in the protein metabolism within the central nervous system (CNS) and these are reflected in an altered composition of the cerebrospinal fluid (CSF). Using two-dimensional gel electrophoresis it is possible to find marker proteins which correlate with the disease. Possible markers of this kind (only an indirect indication of BSE) were first detected in a late stage of the incubation period of experimental BSE. However, it is known from comparative experiments with samples taken from Creutzfeldt-Jakob patients that this marker can also be found in Alzheimer's patients. Alzheimer's disease is regarded as a non-transmissible spongiform encephalopathy.

Even if simpler methods were developed for detection, it is unlikely that such tests would be useful for diagnosing pre-clinical BSE.

The prior art describes methods of preparing synthetic polypeptides with antigen determinants of prion protein (WO 93/11155, WO93/23432), antibodies specific for native Scrapie prion protein (WO97/10505), and methods of detecting Scrapie in sheep (WO97/37227). Korth et al. (1997, Nature 390: 74-77) describe a monoclonal antibody from mice which is able to distinguish between the cellular isoform (PrP^{c}) and the Scrapie isoform (PrP^{sc}).

### DETAILED DESCRIPTION OF THE INVENTION

The aim of the present invention is to provide a method of diagnosing pre-clinical or clinical transmissible spongiform encephalopathies.

This objective has been achieved according to the present invention within the scope of the specification and claims by means of a method of diagnosing pre-clinical or clinical transmissible spongiform encephalopathies.

According to the invention, the method is characterised in that

a) a blood sample is taken from a live mammal

b) cells are concentrated from this blood sample, said cells are referred to as target cells

c) the expression of a marker protein for transmissible spongiform encephalopathies is determined in the target cells

d) the result obtained is compared with a control value.

In a particular embodiment of the method according to the invention the target cells are homogenised.

The pre-clinical phase of transmissible spongiform encephalopathies is the long incubation period after infection with the prion protein with no external clinical symptoms. The present invention makes it possible, in particular, to diagnose transmissible spongiform encephalopathies during this phase with no external clinical symptoms. Thus, the present invention also makes it possible to make a diagnosis in mammals in which transmissible spongiform encephalopathies are suspected but in which the histopathological findings are (still) negative at the time of the test (TSE-suspected, cf. also the description for BSE above). The clinical phase is the brief phase of clinical symptoms which follows the pre-clinical phase and has hitherto invariably led to the death of the infected mammals owing to the absence of any treatment. Transmissible spongiform encephalopathies can also be diagnosed during this phase using the technical teaching of the present invention. The transmissible spongiform encephalopathies (TSE) include in particular Scrapie in sheep, bovine spongiform encephalopathy (BSE) in cattle and Kuru-Kuru disease and Creutzfeldt-Jakob's disease in humans.

Determining the expression of a marker protein means that the said marker protein is demonstrably raised or lowered compared with a control. Demonstrably means, for example, that the marker protein is expressed 50 to 100% higher or lower than in the control or is statistically significantly raised or lowered. A control value or standard can be determined, for example, using cells from non-infected animals and is used to calibrate the method according to the invention. Methods of doing this are known to those skilled in the art.

Marker proteins may be any proteins known to the skilled person which are demonstrably raised or lowered in pre-clinical or clinical transmissible spongiform encephalopathies. This is the case, for example, if the marker protein is undetectable in the control and can clearly be identified using the methods described below in infected mammals or mammals which are suspected of having a transmissible spongiform encephalopathy.

In one particular embodiment the method according to the invention is characterised in that the marker protein is the prion protein PrP-sen. The prion protein PrP-sen according to the invention is the cellular isoform of the prion protein which is often referred to as PrP^{c}. PrP-sen (sen=sensitive) is totally degraded by proteases.

In one particular embodiment the method is characterised in that the marker protein is interferon gamma (IFNγ). In yet another particular embodiment the method is characterised in that the marker protein is bovine interferon gamma (IFNγ). IFNγ (e.g. Vilcek, J. and Oliveira, I. C. Int Arch Allergy Immunol 1994, 104: 311-316) and bovine IFNγ (Keefe, R. G. et al., Vet Immunol Immunopathol, 1997, 56: 39-51) are known to those skilled in the art.

In another particular embodiment the method is characterised in that the marker protein is the laminin receptor (LR) or the laminin receptor precursor (LRP). The laminin receptor (e.g. Grosso, L. E. et al., Biochemistry, 1991, 30: 3346-3350) and the laminin receptor precursor (e.g. Castronovo, V. et al., J Biol Chem 1991, 266: 20440-20446) are known in the art. In another even more particular embodiment the method is characterised in that the marker protein is the bovine laminin receptor (LR) or the bovine laminin receptor precursor (LRP).

The said marker proteins can be detected using any methods known to the average skilled person.

In a preferred embodiment the marker protein is determined by an immune test. An immune test uses monoclonal antibodies or polyclonal antisera specific to the marker protein which are available in the art. For example, the monoclonal antibody l3 or the monoclonal antibody l42 may be used for the marker protein PrP^{sen} (Harmeyer S. et al., J Gen Virol 1998, 79, 937-945, see FIG. 1). Immune tests include the methods of detection known in the art such as the ELISA test (enzyme-linked immuno-sorbent assay) or the so-called sandwich-ELISA test, dot blots, immunoblots, radioimmuno tests (radioimmunoassay RIA), diffusion-based Ouchterlony test or rocket immunofluorescent assays). Another immune test is the so-called Western blot (also known as Western transfer procedure or Western blotting). The purpose of Western blot is to transfer proteins or polypeptides separated by polyacrylamide gel electrophoresis onto a nitrocellulose filter or other suitable carrier and at the same time retain the relative positions of the proteins or polypeptides obtained from the gel electrophoresis. The Western blot is then incubated with an antibody which specifically binds to the protein or polypeptide under consideration. These methods of detection can be used by the average skilled person to perform the invention described herein. Literary references in which the skilled person can find the above-mentioned methods and other detection methods are listed as follows: An Introduction to Radioimmunoassay and Related Techniques, Elsevier Science Publishers, Amsterdam, The Netherlands (1986); Bullock et al., Techniques in Immunocytochemistry, Academic Press, Orlando, Fla. Vol. 1 (1982), Vol. 2 (1983), Vol. 3 (1985); Tijssen, Practice and Theory of Enzyme Immunoassays: Laboratory Techniques in Biochemistry and Molecular Biology, Elsevier Science Publishers, Amsterdam, The Netherlands (1985).

In another, most particular embodiment, the target cells are incubated with antibodies which are specific to the marker protein and the antigen/antibody complex thereby formed is determined.

In a particularly preferred embodiment of the method according to the invention, the altered expression of the marker protein for transmissible spongiform encephalopathies is determined by molecular biology methods. Molecular biology methods as used herein means detection methods which include, for example, polymerase chain reaction (PCR) or may be Northern or Southern blots which the skilled person can find in the standard reference books (e.g. Sambrook et al. (1989) Molecular Cloning: A Laboratory Manual, 2^{nd} ed., Cold Spring Harbor Laboratory Press, Cold Spring Harbor, N.Y. and Bertram, S. and Gassen, H. G. Gentechnische Methoden, G. Fischer Verlag, Stuttgart, N.Y., 1991).

In another preferred embodiment of the method according to the invention the marker protein for transmissible spongiform encephalopathies is determined by a reverse transcriptase polymerase chain reaction (RT-PCR). In this special form of the polymerase chain reaction (PCR) first of all the total RNA is isolated, this is reverse transcribed using the enzyme “reverse transcriptase” into cDNA with which the PCR reaction is then carried out. This detection method is known to those skilled in the art and is published in standard reference books (e.g. Sambrook et al. I1989) Molecular Cloning: A Laboratory Manual, 2^{nd} ed., Cold Spring Harbor Laboratory Press, Cold Spring Harbor, N.Y. and Bertram, S. and Gassen, H. G. Gentechnische Methoden, G. Fischer Verlag, Stuttgart, N.Y., 1991).

Examples of “live mammals” are known to the average skilled person and include, for example, human beings as well as sheep, goats, pigs, cattle, deer, rabbits, hamsters, rats and mice.

In one particular embodiment the method is characterised in that the live mammal is a member of all bovidae family, most preferred a cow or a sheep. Although the application relates particularly to methods of diagnosing transmissible spongiform encephalopathies in cattle, the technical teaching is equally applicable to any animal which can be afflicted with the pathogen of said encephalopathies and is therefore included in the present invention.

The cells contained in the blood sample comprise all the blood cells obtained from a haematopoietic stem cell, e.g. lymphocytes, thrombocytes, platelets or erythrocytes.

In another particular embodiment, the method is characterised in that the target cells are leukocytes. The term leukocytes includes, for example, polymorphonuclear and mononuclear leukocytes, mast cells, B-cells or B-lymphocytes, T-cells or T-lymphocytes and natural killer cells (NK cells).

In a most particular embodiment the method is characterised in that the target cells are mononuclear leukocytes. The term mononuclear leukocytes refers in particular to monocytes and macrophages, dendritic cells and Langerhans cells.

In another particular embodiment the method is characterised in that the target cells are polymorphonuclear leukocytes. The polymorphonuclear leukocytes include the eosinophilic, neutrophilic and basophilic granulocytes.

The invention further relates to a diagnostic test kit for detecting spongiform encephalopathies which contains all the elements required to detect the altered expression of a marker protein for transmissible spongiform encephalopathies using a method according to the invention.

The invention further relates, in particular, to a diagnostic test kit which contains antibodies specific to a marker protein for transmissible spongiform encephalopathies.

The invention further relates, in particular, to a diagnostic test kit, characterised in that the antibodies according to the invention are polyclonal.

The invention further relates, in particular to a diagnostic test kit, characterised in that the antibodies according to the invention are monoclonal.

The invention also includes a diagnostic test kit according to the invention which is characterised in that it contains all the necessary elements for detecting the altered expression of the marker protein PrP-sen by a method according to the invention.

The invention also includes a diagnostic test kit according to the invention which is characterised in that it contains all the necessary elements for detecting the altered expression of the marker protein IFNγ by a method according to the invention.

The invention also includes a diagnostic test kit according to the invention which is characterised in that it contains all the necessary elements for detecting the altered expression of the marker protein bovine IFNγ by a method according to the invention.

The invention also includes a diagnostic test kit according to the invention which is characterised in that it contains all the necessary elements for detecting the altered expression of the marker protein laminin receptor (LR) or the marker protein laminin receptor precursor (LRP) by a method according to the invention.

The invention also includes a diagnostic test kit according to the invention which is characterised in that it contains all the necessary elements for detecting the altered expression of the marker protein bovine laminin receptor (LR) or the marker protein bovine laminin receptor precursor (LRP) by a method according to the invention.

The invention also relates, in particular, to a diagnostic test kit which is suitable for carrying out an immune test in situ.

A diagnostic test kit is a collection of all the components for a method of diagnosis according to the invention. Some examples (not an exhaustive list) of other elements for performing a method according to the invention include containers such as 96-well plates or microtitre plates, test tubes, other suitable containers, surfaces and substrates, membranes such as nitrocellulose filter, washing reagents and buffers. A diagnostic test kit may also contain reagents which may detect bound antibodies, such as for example labelled secondary antibodies, chromophores, enzymes (e.g. conjugated with antibodies) and the substrates thereof or other substances which are capable of binding antibodies.

The invention also relates to a diagnostic test kit for detecting transmissible spongiform encephalopathies, which contains oligonucleotides capable of hybridising under stringent conditions to the nucleic acid coding for a marker protein for transmissible spongiform encephalopathies, and the other elements needed to carry out a method according to the invention.

The invention further relates to a diagnostic test kit according to the invention which is characterized in that it contains all the necessary elements for carrying out a reverse transcriptase polymerase chain reaction (RT-PCR). Said kit may contain, but is not limited to in addition to test tubes or 96-well plates or microtitre plates, other suitable containers, surfaces and substrates, membranes such as nitrocellulose filters, washing reagents and reaction buffers (which may vary in pH and magnesium concentrations), sterile water, mineral oil, BSA (bovine serum albumin), MgCl₂, (NH₄)₂SO₄, DMSO (dimethylsulphoxide), mercaptoethanol, nucleotides (dNTPs), enzymes such as Taq-polymerase and reverse transcriptase and, as the DNA matrix, the DNA sequence of the marker protein or parts thereof, oligonucleotides specific for a marker protein according to the invention, control template, DEPC-water, DNAse, RNAse and further compounds known to the skilled artisan.

Oligonucleotides according to the invention are short nucleic acid molecules from about 15 to about 100 nucleotides long, which bind under stringent conditions to the nucleic acid sequence which is complementary to a marker protein. By stringent conditions the skilled person means conditions which select for more than 85%, preferably more than 90% homology (cf. Sambrook et al. (1989) Molecular Cloning: A Laboratory Manual, 2^{nd} ed., Cold Spring Harbor Laboratory Press, Cold Spring Harbor, N.Y. and Bertram, S. and Gassen, H. G. Gentechnische Methoden, G. Fischer Verlag, Stuttgart, N.Y., 1991).

The invention further relates to a diagnostic test kit according to the invention containing oligonucleotides which are capable of hybridising under stringent conditions with the nucleic acid coding for PrP-sen.

The invention also relates to a diagnostic test kit according to the invention containing oligonucleotides which are capable of hybridising under stringent conditions with the nucleic acid coding for IFNγ.

The invention also relates to a diagnostic test kit according to the invention containing oligonucleotides which are capable of hybridising under stringent conditions with the nucleic acid coding for bovine IFNγ.

The invention also relates to a diagnostic test kit according to the invention containing oligonucleotides which are capable of hybridising under stringent conditions with the nucleic acid coding for the laminin receptor (LR) or the laminin receptor precursor (LRP).

In another embodiment the present invention relates to the use of an antibody which is specific for PrP-sen in a method according to the invention.

In another embodiment the present invention relates to the use of an antibody which is specific for IFNγ in a method according to the invention.

In another embodiment the present invention relates to the use of an antibody which is specific for bovine IFNγ in a method according to the invention.

In another embodiment the present invention relates to the use of an antibody which is specific for the laminin receptor (LR) or the laminin receptor precursor (LRP) in a method according to the invention.

In another preferred embodiment the present invention relates to the use of oligonucleotides which are capable of hybridising under stringent conditions to the nucleic acid coding for PrP-sen in a method according to the invention.

In another preferred embodiment the present invention relates to the use of oligonucleotides which are capable of hybridising under stringent conditions to the nucleic acid coding for IFNγ in a method according to the invention.

In another preferred embodiment the present invention relates to the use of oligonucleotides which are capable of hybridising under stringent conditions to the nucleic acid coding for bovine IFNγ in a method according to the invention.

In another preferred embodiment the present invention relates to the use of oligonucleotides which are capable of hybridising under stringent conditions to the nucleic acid coding for the laminin receptor (LR) or the laminin receptor precursor (LRP) in a method according to the invention.

Another preferred embodiment of the invention is the use of the diagnostic test kit according to the invention for detecting transmissible spongiform encephalopathies in the diagnosis of human and animal spongiform encephalopathies or for epidemiological control measures for endemic BSE or Scrapie.

### BRIEF DESCRIPTION OF THE FIGURES

FIG. 1: Determining the Marker Protein PrP^{sen} in a Western Blot

The Figure shows the determining of the marker protein PrP^{sen} in mononuclear (MN) leukocytes of BSE infected cattle and BSE-negative control animals in a Western blot using l42 monoclonal antibodies.

The cells were homogenised in 2% sarcosyl. The homogenised preparation is applied to the gel in a concentration of 60 μg/well.

Trace 1: Molecular weight marker

Trace 2: BSE brain (BSE positive, positive control)

Trace 3: Cow No. 058193 (BSE positive)

Trace 4: Cow No. 5061 (BSE positive)

Trace 5: Cow No. 2819 (BSE positive)

Trace 6: Cow No. 279046 (BSE negative, negative control)

Trace 7: Cow No. 4751 (BSE positive)

All the MN samples are obtained from BSE infected cattle, with the exception of cow no. 279046. The expression of PrP^{sen} is positive in all the BSE infected cattle compared with the negative control. Cow no. 5061 (trace 4) expresses PrP^{sen} less strongly here but significantly above the negative control.

FIG. 2: Determining the Marker Protein IFN-γ by RT-PCR

The Figure shows the determining of the marker protein IFN-γ by RT-PCR in BSE infected cattle and BSE negative control animals.

Trace 1: GAPDH control, Cow No. 4372 (BSE positive)

Trace 2: IFN-γ, Cow No. 4372 (BSE positive)

Trace 3: GAPDH control, Cow No. 441 (BSE negative)

Trace 4: IFN-γ, Cow No. 441 (BSE negative)

FIG. 3: Determining the Marker Protein Laminin Receptor by RT-PCR

The Figure shows the measurement of the marker protein laminin receptor (LR) by RT-PCR in BSE-infected cattle, cattle in which BSE is suspected and in BSE-negative control animals.

Part A)

Trace 1: Cow No. 4471 (BSE positive)

Trace 2: Cow No. 58193 (BSE positive)

Trace 3: Cow No. 462 (negative control)

Trace 4: Cow No. 5621 (BSE suspected)

Trace 5: Cow No. 5054 (BSE suspected)

Trace 6: Target DNA control

Trace 7: Void

Trace 8: Molecular weight marker

Part B)

For each trace in Part A) there is a corresponding RT-PCR of D-glyceraldehyde-3-phosphate-dehydrogenase (GAPDH) as a control for the differential activity of reverse transcriptase.

The invention is described more fully with reference to the Example which follows.

### EXAMPLE 1

### Diagnosis of BSE by Means of the Increased Expression of Specific Marker Proteins in Isolated Leukocytes

The Example which follows describes the diagnosis of BSE in cattle by determining the increased expression of the marker proteins PrP^{sen} or IFN-γ or the laminin receptor (precursor) (LR(P)) on isolated mononuclear (MN) or polymorphonuclear (PMN) leukocytes.

### Isolation of Mononuclear (MN) and Polymorphonuclear (PMN) Leukocytes from Bovine Whole Blood

These special blood cells are isolated by two centrifugation steps, the latter being a density gradient centrifugation in order to obtain the so-called leukocyte “buffy” coat, followed by lysis of the erythrocytes.

Step 1: Blood Samples

The blood samples (about 400 ml in volume) are taken from the animals and placed directly in a special container. This container is already provided with a mixture of glucose and citrate: 68 mM glucose, 37.4 mM tri-sodium citrate, 17.4 mM citric acid, adjusted to pH 7.3, as anticoagulant. The blood and anticoagulant are in a ratio of 6:1. The blood samples are sent immediately to the laboratory for isolation of the cells.

Step 2: Concentration of Leukocytes

1. Centrifugation

Exactly 40 ml of whole bovine blood treated with anticoagulant is placed in a sealable sterile 50 ml centrifugal test tube and centrifuged at 800 × g for 20 minutes at room temperature in a rotary centrifuge without brake. Centrifugation should be extended for 5 minutes for each hour (up to 3 hours) that the blood samples have been stored after collection.

This first centrifugation leads to the formation of three separate bands:

Upper band—serum

Middle band—leukocytes (so-called “buffy”)

Lower band—erythrocytes

Density Centrifugation Medium:

Standard commercial medium can be used to isolate the leukocytes. We used in NYCOMED Lymphoprep™, density 1.077 g/ml.

The serum is carefully removed and deep-frozen for further analysis at −20° C. The leukocyte layer is taken off and placed in a new centrifugal test tube.

2^{nd} Centrifugation

Exactly 15 ml of the leukocytes from the first centrifugation are placed on 35 ml of NYCOMED Lymphoprep™, density 1.077 g/ml in a sealable sterile 50 ml centrifugal test tube.

Centrifugation: 800× g/20 min-(RT) in a rotary centrifuge without a brake.

Centrifugation should be extended for 5 minutes for each hour (up to 3 hours) that the blood samples have been stored after collection.

This 2^{nd} centrifugation leads to the formation of four separate bands:

From top to bottom:

1^{st} band—(residual) serum

2^{nd} band—mononuclear leukocytes (monocytes and lymphocytes=buffy)

3^{rd} band—medium interface

4^{th} band—polymorphonuclear leukocytes and (residual) erythocytes

Step 3: Separation of the Mononuclear (MN) Leukocytes

Band 2 contains monocytes and lymphocytes and is carefully sucked out using a sterile Pasteur pipette and transferred into a sterile 50 ml centrifugal test tube. The cells are washed twice with the same volume of sterile PBS (phosphate buffered saline) and centrifuged at 600× g/15 min/10° C. The pelleted cells are resuspended in HBSS (Hanks balanced salt solution with NaHCO₃, without phenol red). The vitality and cell number are determined using trypan blue.

Step 4: Separation of the Polymorphonuclear (PMN) Leukocytes

After band 2 has been pipetted out, bands 1 and 3 are also removed by suction. The PMN/erythrocyte mixture is then diluted three times with sterile erythrocyte lysing buffer (ELB, consisting of 8.9 mM KHCO₃, 154.9 mM NH₄CL and 0.01 mM EDTA), mixed carefully and incubated for 10 min at RT.

Centrifugation: 800× g/10 min/10° C.

The supernatant is discarded and the pellet is resuspended in 20 ml of ELB buffer, mixed, incubated and centrifuged again.

Centrifugation: 800× g/10 min/10° C.

The supernatant is discarded and the pellet is washed with 20 ml HBSS (as above, but with the addition of 1 mM MgCl₂).

Centrifugation: 800× g/8 min/10° C.

The supernatant is discarded and the cells are resuspended in HBSS. The vitality and cell number are determined with trypan blue.

Results: Example Cell Numbers

| Cell type | Cell number/ml of blood | Vitality |
|---|---|---|
| 1. MN | 2.25 × 10⁶ | ≧93% |
| 2. PMN | 4.03 × 10⁶ | ≧98% |

The following measurements were made with the isolated MN- and PMN-leukocytes of BSE infected animals:

I. Increased expression of the cellular isoform of the prion protein, PrP^{sen}

II. Increased expression of the interferon gamma protein, IFN-γ

III. Increased expression of the laminin (precursor) receptor, L(P)R

I. Increased Expression of the Cellular Isoform PrP^{sen}

The expression rate of PrP^{sen} in isolated leukocytes from control animals and BSE infected animals is measured by Western blot analysis (Harmeyer S. et al., J Gen Virol 1998, 79, 937-945). Chromogenic development is used.

Opening Up of the Cells:

The isolated leukocytes are homogenised in 2% sarcosyl solution (Sigma, St. Louis, U.S.A.) for 10 min/4° C. The homogenised preparation thus obtained is then pelleted at 15,000× g/40 min/4° C. The supernatant is suction filtered and stored at −20° C.

The protein concentration of the homogenised preparation was determined and all samples were standardized to 6 mg/ml protein. Exactly 60 μg of the homogenate was loaded into each well.

Detection is carried out using either the monoclonal antibody 13 or the monoclonal antibody l42. The antibodies are described in detail in the above-mentioned publication. The dilution of the antibody is 1:10 in each case.

The secondary antibody used is this example were AP-conjugated antibodies in a dilution of 1:3000.

Results

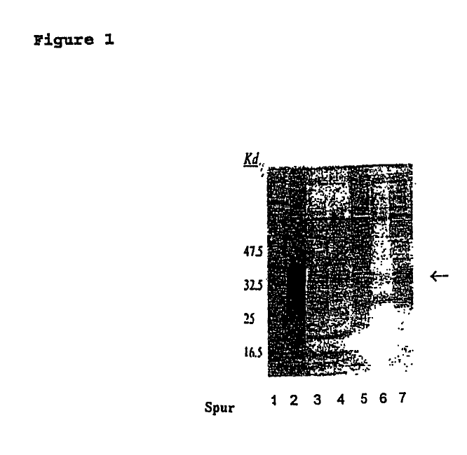

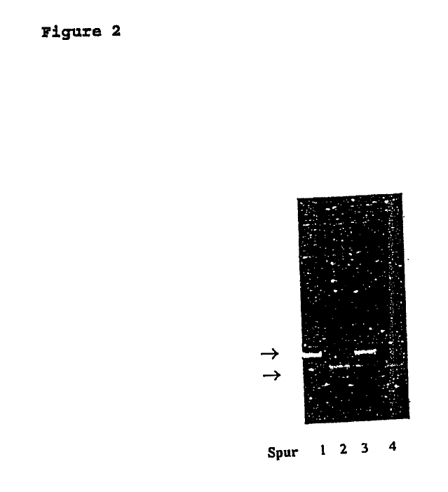

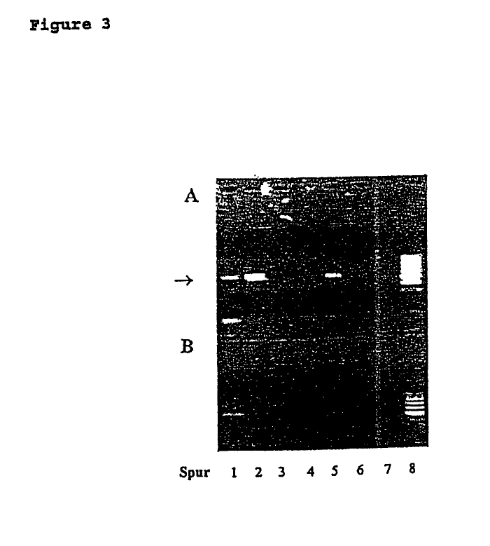

It was shown that the protein expression of PrP^{sen} is significantly raised both in MN- and PMN-leukocytes of BSE infected animals compared with healthy control animals (cf. also the Western blot of FIG. 1 with samples from other cattle).

| Case No./ | | Increased |
|---|---|---|
| Sample | BSE Status | expression of PrP^{sen} |
| 4372 | positive | yes |
| 4471 | positive | yes |
| 4401 | suspect | yes |
| | Negative | |
| Control | negative | no |
| | negative | |

II. Increased Expression of IFN-γ

The increased expression is measured in two ways:

measurement of the protein by ELISA

measurement of the specific mRNA by RT-PCR

1. IFN-γ Measurement Using ELISA

A commercial ELISA made by CSL Veterinary Ltd., Melbourne, Australia is used.

Results:

| BSE Status | Number of animals | IFN-γ (pg/ml) |
|---|---|---|
| BSE positive | 9 | 314.0 ± 78.2 |
| BSE suspected | 9 | 504.0 ± 109.7 |
| BSE negative | 13 | 0.0 ± 0.0 |

2. IFN-γ mRNA Measurement Using RT-PCR

The isolation of RNA (from the total leukocyte fraction) and the subsequent RT-PCR are carried out by standard methods (Yi-Jun Shi and Jing-Zhlong Liu, Genet. Anal. Tech. Appl., 1992, 9, 149-150; Izraeli S. et al., Nucl. Acid. Res. 1991, 21, 6051; Michel U. et al., Anal. Biochem. 1997, 249, 246-247) with the modifications described below.

Isolation of RNA:

The Promega System is used to isolate the total RNA.

cDNA (RT Reaction):

Isolated RNA samples are reverse transcribed using the Reverse Transcription System of Promega (Catalogue No. A3500, Promega Life Sciences 1999 Catalogue, pg. 10.11).

PCR Reaction:

In order to determine the specific mRNA a double polymerase chain reaction (“nested PCR”) is used.

The IFN-γ primers used for this:

| | | |
|---|---|---|
| FW1 | 5′GGAGTATTTTAATGCAAGTAGCCC 3′ | [SEQ ID NO.1] |
| (IFNF1) | | |
| FW2 | 5′GTAGCTAAGGGTGGGCCTCT 3′ | [SEQ ID NO.2] |
| (IFNF2) | | |
| RV | 5′GCTCTCCGGGCCTCGAAAGAGATT 3′ | [SEQ ID NO.3] |
| (IFNR1) | | |

The PCR product to be expected should be 357 base pairs (bp) long.

Results:

The IFNγ ELISA is clearly confirmed by this RT-PCR, i.e. in BSE infected animals IFNγ is significantly raised.

FIG. 2 shows the RT-PCR of mRNA from total leukocytes in whole blood with the samples of cattle nos. 4372 and 441.

III. Increased Expression of the Laminin Receptor (Precursor), LR(P)

The expression was measured by RT-PCR. This reaction also includes the detection of LRP as well as LR.

The bovine sequence of LRP or LR has not yet been described. However, this protein is highly conserved in mammals. The published sequence data of human as well as murine LR are therefore compared. On the basis of this data analysis the following primer sequence is established:

Primers

| | | |
|---|---|---|
| Forwards | 5′ AAGAGGACCTGGGAGAAGCT 3′ | [SEQ ID NO.4] |
| Backwards | 5′ CCTTCTCAGCAGCAGCCCTGC 3′ | [SEQ ID NO.5] |

Expected product: 517 bp

RNA isolation

As described under point II.2.

cDNA (RT reaction)

As described under point II.2.

PCR reaction

Simple reaction corresponding to standard methods.

Results

| Case No./ | | Increased |
|---|---|---|
| Sample | BSE Status | expression of LRP/LR |
| 4471 | positive | yes |
| 58193 | positive | yes |
| 5621 | suspected | (yes) |
| 5054 | suspected | yes |
| Control (462) | negative | no |

These results are shown in FIG. 3.

IV. Cloning and Expression of the Bovine Laminin Receptor (Precursor), LR(P), to Generate Specific Antibodies Against LR.

Development of Primers Towards LR

Primers towards bovine LR are designed to amplify the entire gene of LR (Genebank Accession No: S 37431). Primers were designed from the bovine c10 protein gene (Genebank Accession No: M 64923).

In the LR primers defined below, their restriction sites appear in a box. Based on these restriction sites a direct cloning into E. coli is possible.

The LR designed primers will be used to amplify the LR from total cellular RNA isolated from whole bovine blood.

The sequence for the three primers may be seen below:

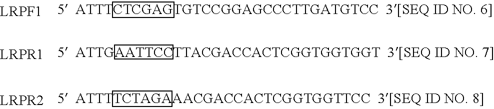

Restriction sites: LRPF1 primer can be cut by Xho, LRPR1 primer can be cut by Eco R1, while LRPR2 can be cut by Xba 1.

RT-PCR and Cloning of Bovine LR Gene:

The LR primers are re-suspended in sterile water to a final concentration of 50 ng/ml.

Bovine leukocyte RNA is isolated from total leukocyte fractions of whole bovine blood.

5 μg of RNA is DNAsed (Gibco BRL) and Reverse Transcription is carried out using a Reverse Transcription Kit (Promega, Cat. No; A3500, Promega Life Sciences 1999 Catalogue, pg. 10.11). Polymerase Chain Reactions (PCR) are carried out using LRPF1, LRPR1, and LRPR2.

PCR: 35 cycles, annealing temperature 50° C.

The amplified fragments obtained following PCR run on 1% Agarose gel to determine fragment size. The resulting bands are gel purified, checked again for size and the fragments are removed from agarose and re-suspended in 10 μl steril water.

The amplified fragments are ligated into pGEM-T vector (Boehringer Mannheim Ligation Kit).

All clones are sequenced and have been demonstrated as being the LR gene.

Sub-cloning into the expression vectors pBAD_{GIII} and pTrcHis (both obtained from Invitrogen Ltd.) has been carried out and the clones are currently being examined for the presence of inserts.

Design of Peptides for the Development of Antibodies to LR:

Four peptides are designed to be utilised in the development of antibodies to the Laminin Receptor (LR).

These peptides are designed using the bovine c10 protein (Genbank Accession No: M 64923; protein Id. AAA62713.1)

A computer program (Antheprot) which predicts a proteins structure, hydrophobic, hydrophiliy and antigenic sites is used for designing the petides.

The principle parameters concentrate upon for the selection of two of the peptides are hydrophilic and antigenic, two other peptides are chosen due to their location at the C- and N-Terminal regions of the protein.

Peptide 1141 MSGALDVLQMKEEDVLKFLAGC [SEQ ID NO. 9]

(Amino Terminal) Corresponding to amino acid residues 1-20 from the amino-terminal end of the protein.

Isoelectric point (pI) of 4.32.

Peptide 1142 RLLVVTDPRADHQPLTEASYGC [SEQ ID NO. 10]

(Antigenic) Corresponding to amino acid residues 120-140 selected from a antigenic region of the Protein.

Isoelectric point (pI) of 5.38.

Peptide 1143 KEEQAAEKAVTKEEFQGEWGC [SEQ ID NO. 11]

(Hydrophilic and antigenic) Corresponding to amino acid residues 212-231 selected from a hydrophilic and antigenic region of the protein.

Isoelectric point (pI) of 4.48.

Peptide 1144 FTAAQPEVADWSEGVQVPSVGC [SEQ ID NO. 12]

(Carboxy Terminal) Corresponding to amino acid residue 238-257 selected from the caraboxy terminal region of the protein.

Isoelectric point (pI) of 3.58.

Each peptide is conjugated to Imject® Maleimide Activated Ovalbumin Carrier Protein (Pierce Warner Ltd.) as follows.

The peptides are dissolved to a final concentration of 10 mg/ml in 100 mM Na₂HPO₄ (pH 7.2).

Imject® Maleimide Activated Ovalbumin is dissolved to a final concentration of 10 mg/ml in steril water.

The peptide and Ovalbumin are allowed to conjugate for 2 hours at room temperature. Following conjugation the conjugated protein solution will be dialyzed in 500-fold volume of PBS (pH 7.4) and stored at −20° C. until required.

Production of Polyclonal and Monoclonal Antibodies

The production of polyclonal and monoclonal antibodies are carried out in a similar way as described e.g., Harmeyer S. et al., J Gen Virol, 1998.

Briefly:

Day 0: Pre-immune bleeds are taken prior to injection for examination of anti-LR-antibodies.

Day 0: 0.5 mls of conjugated peptide suspended in Freund's Complete Adjuvant are injected subcutaneous.

Day 14: the first booster injection of 0.5 mls conjugated peptide in Incomplete Freund's Adjuvant.

Day 21: the second booster injection of 1.0 ml conjugated peptide in the absence of adjuvant.

Day 31: Test Bleed for control of produced antibodies.

ELISA System for the Measurement of Marker-Proteins

The ELISA technology is especially suitable for the measurement of BSE marker-proteins. In this case special blood cells will be separated which carry these markers on the cell surface.

Cell Separation Procedure

Sub-division of MN and PMN leukocyte sub-classes (target cells) will be achieved using immunomagnetic bead (Dynabeads®) depletion. Beads will be coated with antibodies specific to bovine leukocyte cell types.

Shortly:

Add cell specific Dynabeads to the blood sample.

Immuncapture of the target cells.

Magnetic separation of the target cells.

Washing and concentration of pure target cells.

ELISA Measurement of Marker-Proteins

An ELISA system is chosen, based upon isolation of target cells using target cell specific capture antibodies:

Microtitre plates are coated with primary capture antibody specific to target cell surface marker.

Incubation with cells expressing target cell surface marker. Binding to primary capture antibody.

Following cell capture, incubation with biotinylated secondary antibody directed against the BSE marker-protein.

Incubation with enzyme-conjugated detection protein. Addition of substrate and measurement by ELISA-reader.

What is claimed is:

1. A method of diagnosing transmissible spongiform encephalopathies, which comprises the steps of:
a) taking a blood sample from a live mammal;
b) concentrating polymorphonuclear leukocytes in the blood sample, said polymorphonuclear leukocytes being referred to as target cells;
c) determining expression of a marker protein for transmissible spongiform encephalopathies in the target cells, wherein said marker protein is the cellular isoform of the prion protein PrP; and
d) comparing the value obtained for the expression of said marker protein with a control value;
wherein an increase in the expression of said marker protein is diagnostic for transmissible spongiform encephalopathies.

2. The method according to claim 1, characterised in that the target cells are homogenised.

3. The method according to claim 1, characterised in that the marker protein expression is determined by an immune test.

4. The method according to claim 1, characterised in that the target cells are incubated with antibodies which are specific to the marker protein and the antigen/antibody complex thereby formed is determined.

5. The method according to claim 1, characterised in that the altered expression of the marker protein for transmissible spongiform encephalopathies is determined by molecular biology methods.

6. The method according to claim 5, characterised in that the altered expression of the marker protein for transmissible spongiform encephalopathies is determined by a reverse transcriptase polymerase chain reaction (RT-PCR).

7. The method according to claim 1, characterised in that the live mammal is a cow.
